(19) United States
(12) Patent Application Publication
(10) Pub. No.: US 2024/0157086 A1
(43) Pub. Date: May 16, 2024

(54) MANUFACTURING METHOD FOR CATHETER AND MANUFACTURING APPARATUS FOR CATHETER

(71) Applicant: TERUMO KABUSHIKI KAISHA, Tokyo (JP)

(72) Inventors: Yuuji MOTOSE, Hiratsuka (JP); Tsutomu SUGIKI, Hadano (JP)

(73) Assignee: TERUMO KABUSHIKI KAISHA, Tokyo (JP)

(21) Appl. No.: 18/417,573

(22) Filed: Jan. 19, 2024

Related U.S. Application Data
(63) Continuation of application No. PCT/JP2022/028476, filed on Jul. 22, 2022.

(30) Foreign Application Priority Data
Aug. 3, 2021 (JP) 2021-127512

(51) Int. Cl. A61M 25/00

(52) U.S. Cl. CPC A61M 25/0009; A61M 2207/10

(57) ABSTRACT

A method for manufacturing a catheter in which a catheter body is formed by fusing a first member and a second member. The method includes irradiating an adjacent portion between the first member and the second member with a laser beam in a close state and in an external force application state to fuse the adjacent portion. In the close state, an elastically deformable elastic body having laser transmission property and having a hollow portion through which the first member and the second member are insertable with a gap at no load is brought into contact with the first member and the second member in the hollow portion due to elastic deformation to bring the first member and the second member close to each other.

### CROSS-REFERENCES TO RELATED APPLICATIONS

This application is a continuation of International Patent Application No. PCT/JP2022/028476 filed on Jul. 22, 2022, which claims priority to Japanese Patent Application No. 2021-127512 filed on Aug. 3, 2021, the entire content of both of which is incorporated herein by reference.

### TECHNOLOGICAL FIELD

The present invention generally relates to a manufacturing method for a catheter and a manufacturing apparatus for a catheter.

### BACKGROUND DISCUSSION

When various therapeutic actions are performed in an organ of a living body, a medical device including a tubular body constituted by a flexible tubular hollow member is often used. Commonly known devices of this type of medical device include a guiding catheter used for delivering a catheter device such as a balloon catheter to a desired position in a living body, a catheter for angiography used for discharging a contrast medium into a living body, and a microcatheter used for discharging a medicine.

The above-described catheter may be manufactured through a step for joining hollow tubes by a laser or the like. The above-described conventional technique of joining tubes to each other at a portion where the tubes are to be joined may employ a heat shrinkable tube. An example is disclosed in Japanese Patent Application Publication No. 2002-301160 (JP 2002-301160 A).

### SUMMARY

The heat shrinkable tube described above shrinks by application of heat in a state of being disposed outside the tubes to be joined, thereby maintaining a state in which the tubes which constitute a product are in contact with each other. Here, the method using the heat shrinkable tube needs a work for removing (detaching) the heat shrinkable tube from the tubes which constitute a product after the joint portion is formed by laser or the like. The present inventors have focused on the fact that the heat shrinkable tube has a relatively large influence on the cost and needs to be discarded after single use. The present inventors have also focused on the fact that an operator who performs a removal work for removing the shrunk tube from the product may not be able to successfully remove the shrunk tube from the product during the removal work and may need to redo the removal work, which leads to an increase in cost.

Disclosed here is a catheter formed by fusing a first member and a second member such as tubes to each other, enable fusing a plurality of times while eliminating the need for a removal work of removing a member required for manufacture from a product after the first member and the second member are fused to each other.

Results such as those discussed above are achieved by virtue of the following.

(1) A manufacturing method for a catheter including a catheter body formed by fusing a first member and a second member, the manufacturing method including irradiating an adjacent portion between the first member and the second member with a laser beam to fuse the adjacent portion in a close state and in an external force application state, the close state indicating a state in which an elastically deformable elastic body having laser transmission property and having a hollow portion through which the first member and the second member are insertable with a gap in a no load state is brought into contact with the first member and the second member in the hollow portion due to elastic deformation to bring the first member and the second member close to each other, the external force application state indicating a state in which a component that has the laser transmission property and that is more rigid than the elastic body applies an external force to the elastic body to suppress a decrease in force due to the elastic deformation for bringing the first member and the second member close to each other.

(2) The manufacturing method for a catheter according to (1), wherein at least one of the first member and the second member includes a material that absorbs a laser beam in the adjacent portion.

(3) The manufacturing method for a catheter according to (1) or (2), wherein the first member and the second member have a tubular shape, and the second member is disposed on a proximal side in an axial direction with respect to the first member.

(4) The manufacturing method for a catheter according to (3), wherein the first member is close to the second member at one end in the axial direction, and the laser beam is applied to an end of the first member different from the adjacent portion in the axial direction.

(5) The manufacturing method for a catheter according to (1), wherein the first member and the second member have a tubular shape, and the component includes a first component that is disposed outside the elastic body in a radial direction intersecting an axial direction of the first member and the second member and applies the external force to the elastic body in the external force application state.

(6) The manufacturing method for a catheter according to (1), wherein the component includes an insertion portion through which the elastic body is insertable, and the elastic body has an outer surface in an insertion direction of the elastic body, the outer surface being formed to have an interference fit with the insertion portion of the component in the no load state.

(7) The manufacturing method for a catheter according to (6), wherein the first member and the second member have a tubular shape, and the elastic body is placed inside the insertion portion of the component with an outer diameter of the elastic body being changed to be smaller than an inner diameter of the component by being extended in an axial direction of the first member and the second member.

(8) The manufacturing method for a catheter according to (1), wherein the component includes a plurality of components distributed in a circumferential direction in the external force application state, and the plurality of components applies the external force to the elastic body toward central axes of the first member and the second member to elastically deform the elastic body in the external force application state.

(9) The manufacturing method for a catheter according to (5), wherein the component includes a second hard component that is disposed outside the elastic body in the axial direction of the first member and the second member and applies the external force to the elastic body.

(10) The manufacturing method for a catheter according to (9), wherein the no load state is shifted to the external force application state by decreasing an internal volume defined by the first component and the second component.

(11) A manufacturing apparatus for a catheter, the manufacturing apparatus including: an insertion member insertable into the first member and the second member; and the elastic body and the rigid component according to any one of (1) to (10).

The manufacturing method for a catheter and the manufacturing apparatus for a catheter according to one aspect of the disclosure enable, in a case where a catheter is formed by fusing a first member and a second member such as tubes to each other, fusing a plurality of times while eliminating the need for a removal work of removing a member required for manufacture from a product after the first member and the second member are fused to each other.

According to another aspect, a manufacturing method for manufacturing an elongated medical device comprises disposing a tubular member in a hollow portion of an elastic body, wherein the tubular body extends in an axial direction from one axial end of the tubular member to an opposite axial end of the tubular member, and wherein the elastic body has a laser transmission property. The disposing of the tubular member in the hollow portion of the elastic body includes disposing the tubular member in the hollow portion of the elastic body so that an end portion of the tubular member, inclusive of the one axial end of the tubular member, is located in the hollow portion of the elastic body. The method additionally includes applying an external force to the elastic body by at least one of two components, with the two components being a first component and a second component that are both positioned outside the elastic body. The first component is positioned radially outside the elastic body in a radial direction that intersects the axial direction, the second component is positioned axially outside the elastic body in the axial direction, the first component having laser transmission property, the at least one of the two components that applies the external force to the elastic body being in contact with the elastic body during the applying of the external force. The method also involves emitting a laser beam through the first component and the elastic body to the distal portion of the tubular member that is located in the hollow portion of the elastic body to deform the distal portion of the tubular member, with the laser beam having a wavelength that generates heat by radiation heating in the distal portion of the tubular member.

According to another aspect, a manufacturing method for manufacturing an elongated medical device includes positioning an elastic body and a tubular member relative to one another so that a distal portion of the tubular member is located in a hollow portion of the elastic body and an outer surface of the tubular member faces an inner surface of the elastic body, wherein the elastic body has a laser transmission property and the tubular member extends in an axial direction. The method also includes positioning a component relative to the elastic body so that an inner surface of the component faces an outer surface of the distal portion of the tubular member, wherein the component has a laser transmission property. The method further incudes applying an external force to the elastic body to press the inner surface of the elastic body to the outer surface of the distal portion of the tubular member, and irradiating the distal portion of the tubular member with a laser beam by virtue of the laser beam passing through the component and the elastic body, wherein the irradiating of the distal portion of the tubular member is performed to generate heat by radiation heating in the distal portion of the tubular member.

### BRIEF DESCRIPTION OF THE DRAWINGS

FIG. 1 is a schematic diagram illustrating an example of a catheter manufactured by a manufacturing method for catheters according to one embodiment disclosed by way of example.

FIG. 2 is a schematic diagram illustrating an example of a manufacturing apparatus for catheters used for a manufacturing method for catheters according to a first embodiment.

FIG. 3 is a schematic diagram illustrating an outer surface and an inner surface of an elastic body constituting the manufacturing apparatus for catheters.

FIG. 4 is a flowchart illustrating the manufacturing method for catheters according to one embodiment disclosed by way of example.

FIG. 5 is a diagram illustrating a state in which a first member and a second member are attached to an insertion member constituting the manufacturing apparatus for catheters.

FIG. 6 is a diagram illustrating a state in which an adjacent portion between the first member and the second member is placed in a hollow portion of the elastic body constituting the manufacturing apparatus for catheters.

FIG. 7 is a diagram illustrating a state in which a rigid component constituting the manufacturing apparatus for catheters is brought close to the elastic body to apply an external force to the elastic body.

FIG. 8 is a diagram illustrating a state in which the adjacent portion between the first member and the second member is irradiated with a laser beam.

FIG. 9 is a schematic diagram illustrating sections of the first member and the second member irradiated with the laser beam.

FIG. 10 is a schematic diagram illustrating a case where the length of a rigid component in the axial direction is shorter than the length of an elastic body in the axial direction according to a modification of the first embodiment.

FIG. 11 is a schematic diagram illustrating a manufacturing apparatus for catheters used in a manufacturing method for catheters according to a second embodiment, and illustrating a state before components constituting a rigid component apply an external force to an elastic body.

FIG. 12 is a diagram illustrating a state in which the components of the manufacturing apparatus for catheters illustrated in FIG. 11 apply an external force to the elastic body.

FIG. 13 is a flowchart illustrating a manufacturing method for catheters according to a modification of the second embodiment.

FIG. 14 is a diagram illustrating a state in which a second rigid component is brought into contact with an elastic body in the manufacturing method for catheters according to the modification of the second embodiment.

FIG. 15 is a diagram illustrating a state in which an adjacent portion between a first member and a second member is placed in a hollow portion of the elastic body in the manufacturing method for catheters according to the modification of the second embodiment.

FIG. 16 is a diagram illustrating a state in which a first rigid component is brought into contact with the elastic body to apply an external force to the elastic body in the manufacturing method for catheters according to the modification of the second embodiment.

FIG. 17A is a diagram illustrating an internal volume defined by the first rigid component and the second rigid component in the state illustrated in FIG. 15 in the modification of the second embodiment; FIG. 17B is a diagram illustrating an internal volume defined by the first rigid component and the second rigid component in the state illustrated in FIG. 16 in the modification of the second embodiment.

FIG. 18 is a diagram illustrating a state in which the adjacent portion between the first member and the second member is irradiated with a laser beam in the manufacturing method for catheters according to the modification of the second embodiment.

FIG. 19 is a schematic diagram illustrating a modification of the first member and the second member.

FIG. 20 is a schematic diagram illustrating a modification of an outer surface of the elastic body.

FIG. 21 is a schematic diagram illustrating a modification of an outer surface of the elastic body.

FIG. 22 is a schematic diagram illustrating a modification of an inner surface of the elastic body.

FIG. 23 is a schematic diagram illustrating a modification of an inner surface of the elastic body.

### DETAILED DESCRIPTION

Hereinafter, an aspect for carrying out the catheter manufacturing method and catheter manufacturing apparatus will be described in detail with reference to the drawings. Embodiments herein are illustrated to embody examples of the new catheter manufacturing method and catheter manufacturing apparatus disclosed here and do not limit the present invention. Furthermore, other aspects, examples, operation techniques, and the like, that could be conceived of by those skilled in the art without departing from the gist of the new catheter manufacturing method and catheter manufacturing apparatus are all included in the scope of the present invention and included in the scope of the claims and equivalents thereof.

Moreover, for convenience of illustration and ease of understanding, the accompanying drawings may be schematically represented by changing a scale, an aspect ratio, a shape, and the like, from actual ones as appropriate, but are merely examples, and do not limit the interpretation of the disclosure here.

In the following description, ordinal numerals such as “first” and “second” will be given, but are used for convenience and do not define any order unless otherwise specified.

A catheter 100 manufactured by the manufacturing method for catheters according to the present embodiment may be inserted into a blood vessel, a bile duct, a trachea, an esophagus, a urethra, or other cavities or lumens in a living body to be used for treatment, diagnosis, or the like. The catheter 100 is a balloon catheter, a microcatheter, a catheter for angiography, a guiding catheter, or the like used for percutaneous transluminal coronary angioplasty (PTCA) or percutaneous transluminal angioplasty (PTA).

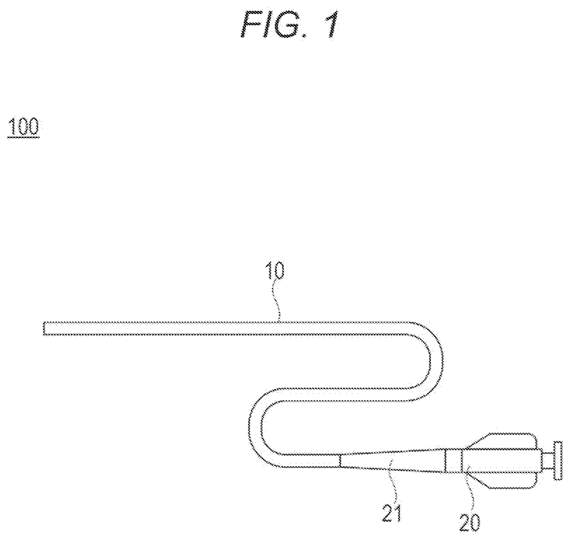

FIG. 1 is a schematic diagram illustrating an example of the catheter 100 manufactured by the manufacturing method for catheters according to an embodiment. Briefly describing the catheter 100 with reference to FIG. 1, the catheter 100 includes an elongated catheter body 10 that can be introduced into a living body and a hub 20 connected to a proximal portion of the catheter body 10. In the present embodiment, the catheter body 10 is configured such that a kink protector 21 is provided in the vicinity of a connecting portion between the catheter body 10 and the hub 20, but the kink protector need not necessarily be provided.

In the present specification, a side of the catheter body 10 on which the hub 20 is disposed is referred to as a proximal side, a side located opposite to the proximal side and introduced into the living body is referred to as a distal side, and a direction in which the tube body extends is referred to as an axial direction.

The catheter body 10 is configured as a flexible tubular member in which a lumen extending in the axial direction is formed. As will be described later in the present embodiment, the catheter body 10 is formed by fusing an adjacent portion P between two tubular members, which are a first member 30 and a second member 40, in a state where the first member 30 and the second member 40 are arranged in the axial direction (see FIG. 9 and the like). The adjacent portion P is provided at one end of the first member 30 in the axial direction.

The first member 30 and the second member 40 have a tubular shape, and the tubular shape is a cylinder as an example in the present embodiment. The second member 40 is located on the proximal side in the axial direction with respect to the first member 30.

Examples of the constituent material from which the first member 30 and the second member 40 may be fabricated include, in addition to a polyamide resin, a polyester resin, a polyolefin resin, and a polyurethane resin, a polyamide elastomer, a polyester elastomer, a polyurethane elastomer, or a mixture of two or more thereof or a mixture of two or more thereof having different rigidity. The first member 30 and the second member 40 preferably include the same kind of constituent materials. As an example, a constituent material of the first member 30 is a polyamide elastomer, and a constituent material of the second member 40 is a polyamide resin. As another example, a constituent material of the first member 30 is a polyester elastomer, and a constituent material of the second member 40 is a polyester resin.

These elastomers may be formed by arranging elastomers having different rigidities so as to be flexible from the proximal end toward the distal end. In addition, the constituent materials of the first member 30 and the second member 40 may contain a polytetrafluoroethylene resin in order to enhance the slidability of the inner surface.

The first member 30 and the second member 40 can contain a pigment or dye that develops white, black, blue, red, or yellow, and a mixture thereof. Such a pigment or dye may be selected from materials that absorb a laser beam and generate heat. Examples of the material that generates heat by absorbing a laser beam include carbon black.

The first member 30 and the second member 40 can be configured to include a powdered radiopaque material. Specific examples of the material include compounds of gold, titanium, bismuth, and tungsten. Furthermore, in the first member 30 and the second member 40, a reinforcing body made of tungsten, SUS, or the like may be disposed in the above-described material. Examples of a mode of the reinforcing body include a coil shape and a blade shape.

The hub 20 is liquid-tightly fixed to the catheter body 10 with an adhesive, a fixing tool (not illustrated), or the like. The hub 20 functions as a port through which a guide wire is inserted into the lumen of the catheter body 10, or an injection port through which a liquid medicine, an embolic substance, a contrast medium, or the like is injected into the lumen, and also functions as a grip portion when the catheter 100 is operated. Examples of a material usable for the hub 20 include a thermoplastic resin such as polycarbonate, polyamide, polysulfone, or polyarylate.

When the catheter 100 has the kink protector 21 as illustrated in FIG. 1, the kink protector 21 can be made of an elastic material provided so as to surround a part of the proximal portion of the catheter body 10. As a constituent material from which the kink protector 21 may be fabricated, natural rubber, silicone resin, or the like can be used, for example.

(Manufacturing Apparatus for Catheters)

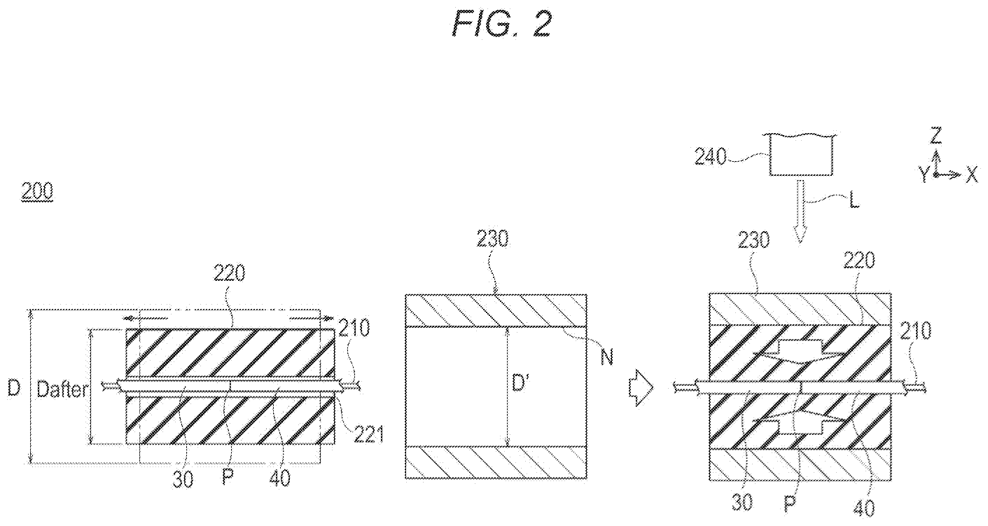

Next, a manufacturing apparatus 200 for the catheter 100 according to the first embodiment will be described. FIG. 2 is a schematic diagram illustrating the manufacturing apparatus 200 for catheters used for the manufacturing method for the catheter 100 according to the first embodiment. Briefly describing the manufacturing apparatus 200 for the catheter 100 with reference to FIG. 2, the manufacturing apparatus 200 includes an insertion member 210, an elastic body 220, a rigid component 230 (corresponding to a first rigid component), and a laser irradiation unit 240.

In the following description, a coordinate system is illustrated in the drawings illustrating the manufacturing apparatus for catheters. X in the orthogonal coordinate system represents an axial direction of the first member 30 and the second member 40 constituting the catheter 100 and is referred to as axial direction X. Y and Z indicate planes intersecting the axial direction X and are referred to as a planar direction YZ. The manufacturing apparatus 200 will be described below in detail.

(Insertion Member)

The insertion member 210 is also called a mandrel and is configured to be insertable into the tubular first member 30 and second member 40 to be fused. In the present embodiment, the insertion member 210 has a circular cross-sectional shape intersecting the axial direction X.

However, the cross section of the insertion member 210 is not limited to have a circular shape, as long as the insertion member 210 can be inserted into the first member 30 and the second member 40, and the cross section may have other shapes such as a polygonal shape. The insertion member 210 may have a tubular shape. The insertion member 210 can be made of a metal material or the like. In addition, the insertion member 210 is rotatably supported by a motor, a gear, a bearing, or the like at both ends or in the vicinity of both ends of the insertion member 210 with the axial direction X as a rotation axis so as to form a fusion portion in a circumferential direction intersecting the axial direction X of the first member 30 and the second member 40.

(Elastic Body)

The elastic body 220 is configured to be elastically deformable so that the first member 30 and the second member 40 to be fused can be brought close to each other. The elastic body 220 is configured to be elastically deformable so as to apply, to an object to be fused such as the first member 30 and the second member 40, a force toward the inside of the object to be fused in the radial direction. While the elastic body 220 maintains a predetermined shape when no load is applied, the elastic body can be deformed toward an unrestrained portion when an external force is applied. The elastic body 220 includes a hollow portion 221 (see FIG. 6). The hollow portion 221 is configured to allow the first member 30 and the second member 40 attached to the insertion member 210 to be inserted therethrough with a gap at no load.

The elastic body 220 is configured to have an interference fit with the rigid component 230 described later. In addition, in the present embodiment, in order to easily insert the elastic body 220 described above into the rigid component 230, jigs (not illustrated) that extend along the elastic body 220 in the axial direction X can be attached to both ends of the elastic body 220 in the axial direction X. As a result, an outer diameter D of the elastic body 220 can be changed to, for example, an outer diameter Dafter illustrated in FIG. 2 that is smaller than an inner diameter D′ of the rigid component 230.

The elastic body 220 is configured to be elastically deformable so as to come into contact with the first member 30 and the second member 40 so the first member 30 and the second member 40 are urged closer to each other in a state where an external force is applied by the rigid component 230 described later. In the present specification, a state in which the first member 30 and the second member 40 are urged closer to each other by the rigid component is referred to as a close state. In addition, in the present specification, a state in which the rigid component applies an external force to the elastic body so that the elastic body is displaced toward the object to be fused is referred to as an external force application state. The external force application state includes, for example, a case where rigid components are disposed outside in a plurality of directions of the elastic body to apply pressure on the elastic body by one rigid component and to prevent or restrict the deformation of the elastic body in a direction other than the direction toward the object to be fused by another rigid component.

The elastic body 220 includes a material having laser transmission properties so that the adjacent portion P between the first member 30 and the second member 40 can be fused when a laser beam is emitted in a state where the elastic body 220 is positioned outside the first member 30 and the second member 40. In the present specification, the laser transmission properties mean that the elastic body is made of a material having a transmittance of 80% or more with respect to laser beam per 1 mm of the thickness of the elastic body in the radial direction.

The elastic body 220 includes a material having heat resistance higher than that of the first member 30 and the second member 40 which are workpieces at the time of fusion between the first member 30 and the second member 40. Examples of the material of the elastic body 220 include silicone rubber and fluororubber.

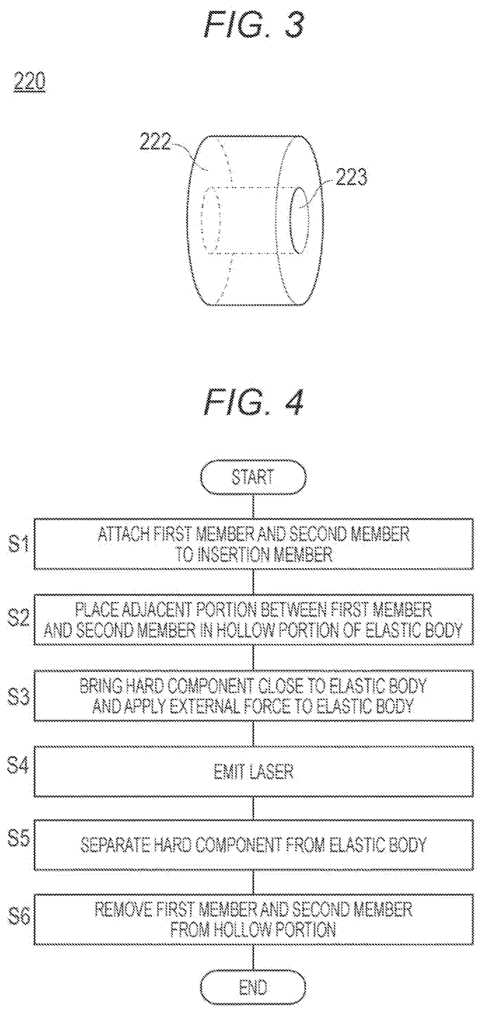

FIG. 3 is a diagram illustrating an outer surface 222 and an inner surface 223 of the elastic body 220. The elastic body 220 includes the outer surface 222 that comes into contact with the rigid component 230 in the external force application state and the inner surface 223 that comes into contact with the first member 30 and the second member 40 in the close state. In the present embodiment, both the outer surface 222 and the inner surface 223 are configured to have a cylindrical side surface shape as illustrated in FIG. 3.

When the cross section of the hollow portion 221 orthogonal to the axial direction X is a circle as illustrated in FIG. 3, the diameter of the hollow portion 221 in a state where no external force is applied is larger than the diameters of the first member 30 and the second member 40. In addition, similar to the insertion member 210, the elastic body 220 is rotatably supported with the axial direction X as a rotation axis.

(Rigid Component)

The rigid component 230 is made of a material more rigid than the elastic body 220. The rigid component 230 is made of a material that is not substantially deformed even when an external force is applied to the elastic body 220 so as to apply a radially inward force to the object to be fused. The rigid component 230 is configured to be able to apply an external force to the elastic body 220 from the outside of the elastic body 220 such that the first member 30 and the second member 40 are brought close to each other by the elastic body 220. The rigid component 230 is configured to be able to apply an external force to the elastic body 220 so as to apply a radially inward force to the object to be fused, such as the first member 30 and the second member 40, by the elastic body 220.

The rigid component 230 limits a decrease in force due to elastic deformation of the elastic body 220 that urges the first member 30 and the second member 40 closer to each other in the external force application state. In the present embodiment, in the external force application state, the rigid component 230 is disposed outside the elastic body 220 in the radial direction intersecting the axial direction X and applies an external force to the elastic body 220 in the radial direction.

In the present embodiment, the rigid component 230 is configured to include a hollow tubular member. As illustrated in FIG. 2, the rigid component 230 includes an insertion portion N as a cavity through which the elastic body 220 can be inserted. The elastic body 220 is configured such that the outer surface in the insertion direction at no load has an interference fit with respect to the insertion portion N of the rigid component 230 as described above. In other words, the outer diameter D (see the alternate long and two short dashes line in FIG. 2) of the outer surface of the elastic body 220 when no load is applied is larger than the inner diameter D′ of the rigid component 230 as illustrated in FIG. 2.

The elastic body 220 can be disposed inside the insertion portion N of the rigid component 230 in a state where the outer diameter D is changed to the outer diameter Dafter that is smaller than the inner diameter D′ of the rigid component 230 due to extension of the elastic body 220 in the axial direction X as described above. In the present embodiment, the length of the rigid component 230 in the axial direction X is set to be the same as the length of the elastic body 220 in the axial direction X as illustrated in FIG. 2 and the like.

Although the rigid component 230 includes the tubular member in the above description, a specific shape of the rigid component is not limited to a hollow tubular member as long as the rigid component 230 can apply an external force to the elastic body 220 so that the first member 30 and the second member 40 are brought close to each other at the fusion portion.

The adjacent portion P between the first member 30 and the second member 40 is irradiated with a laser beam with the rigid component 230 being disposed outside the first member 30, the second member 40, and the elastic body 220. To this end, similar to the elastic body 220, the rigid component 230 is configured to have laser transmission properties. Similar to the elastic body 220, the laser transmission properties of the rigid component 230 mean that the rigid component is configured to have transmittance of 80% per 1 mm of the thickness of the elastic body in the radial direction.

A specific material of the rigid component 230 is not particularly limited as long as the rigid component 230 can deform the elastic body 220 into a desired shape, is more rigid (has higher/greater rigidity) than the elastic body 220 to such an extent that the rigid component 230 is not substantially deformed with respect to a reaction force from the elastic body 220, and can transmit a laser beam. Examples of the specific material of the rigid component 230 include glass, quartz, sapphire, or a material having higher/greater rigidity than the elastic body 220 among the materials mentioned for the elastic body 220. The rigid component 230 can be referred to as an external force applying member. In addition, similar to the insertion member 210 and the elastic body 220, the rigid component 230 is rotatably supported with the axial direction X as a rotation axis.

(Laser Irradiation Unit)

The laser irradiation unit 240 is used to fuse the adjacent portion P between the first member 30 and the second member 40. The laser irradiation unit 240 includes a light source (not illustrated) that oscillates a laser, a galvano scan (scanner) that changes the laser oscillated from the light source in a predetermined direction by a motor, a mirror, or the like, a prism, and the like.

The laser irradiation unit 240 emits a laser beam having a wavelength that causes the fusion portion to generate heat by radiation heating. The spot diameter of the laser beam can be set to φ0.1 to φ10 mm, and the wavelength of the laser beam can be set to 800 to 10000 nm. Thus, the laser beam from the laser irradiation unit 240 passes through the rigid component 230 and the elastic body 220, and is applied toward the adjacent portion P between the first member 30 and the second member 40, so that a fusion portion can be formed. The adjacent portion irradiated with the laser beam is an end face portion of the two members (the first member 30 and the second member 40) and a peripheral portion thereof in a state where both members are arranged in the axial direction (i.e., in a state in which the two members are axially adjacent each other as shown in FIG. 2). Alternatively, the adjacent portion irradiated with the laser beam is an overlapping portion or a peripheral portion thereof in a state where the two members overlap each other.

(Manufacturing Method for Catheters)

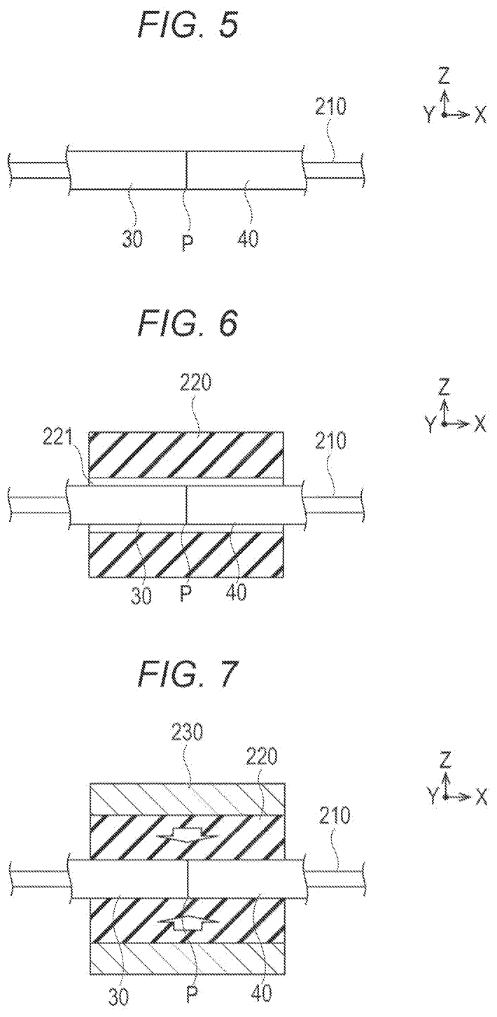

Next, the manufacturing method for the catheter 100 according to the present embodiment will be described. FIG. 4 is a flowchart illustrating the manufacturing method for the catheter 100 according to the present embodiment, and FIGS. 5 to 9 are diagrams for describing the manufacturing method for the catheter 100 according to the present embodiment.

The manufacturing method for the catheter 100 according to the present embodiment will be briefly described with reference to FIG. 4. The manufacturing method includes attaching the first member 30 and the second member 40 to the insertion member 210 (S1), and placing the first member 30 and the second member 40 in the hollow portion 221 of the elastic body 220 (S2). The method also includes applying an external force to the elastic body 220 by the rigid component 230 (S3), emitting a laser beam (S4), separating the rigid component 230 from the elastic body 220 (S5), and removing the first member 30 and the second member 40 (S6) from the elastic body. The manufacturing method will be described below in detail.

First, the insertion member 210 is inserted into the first member 30 and the second member 40 (S1) as illustrated in FIG. 5. In this state, the first member 30 and the second member 40 are integrally movable and rotatable with the insertion member 210. FIG. 5 illustrates the state in which end faces of the first member 30 and the second member 40 facing each other are in contact with each other. However, a state in which the first member 30 partially overlaps (axially overlaps) the second member 40 (FIG. 19) or a state in which a gap is formed between the first member 30 and the second member 40 as described later may also be included.

Next, in a state where the rotation axes of the insertion member 210 and the elastic body 220 are aligned with each other, one of the insertion member 210 and the elastic body 220 is moved in the axial direction X to place the adjacent portion P between the first member 30 and the second member 40 in (within) the hollow portion 221 of the elastic body 220 (S2). In this state, the first member 30 and the second member 40 are not in contact with the hollow portion 221 of the elastic body 220 in the radial direction, and there is a gap therebetween, as illustrated in FIG. 6.

Next, as illustrated in FIG. 7, the rigid component 230 is brought close to the elastic body 220 to apply an external force to the elastic body 220. In the present embodiment, both end portions (or the vicinity of both end portions) of the elastic body 220 in the axial direction X are gripped and extended in the axial direction X to change (reduce) the outer diameter D of the elastic body 220 to the outer diameter Dafter as illustrated in FIG. 2, and with this state, the elastic body 220 is disposed inside the insertion portion N of the rigid component 230. By temporarily reducing the outer diameter of the elastic body 220 in this manner, the elastic body 220 can be easily inserted into the rigid component 230.

After the elastic body 220 is disposed inside the insertion portion N of the rigid component 230, the outward extension of the elastic body 220 in the axial direction X is released. Thus, the outer diameter Dafter (outer surface) of the elastic body 220 is displaced radially outward, so that the inner surface of the rigid component 230 meets the outer surface of the elastic body 220, and an external force is applied radially inward to the elastic body 220 by the rigid component 230 (S3). While the outer surface 222 of the elastic body 220 is in close contact with the inner surface of the rigid component 230, the elastic body 220 receives a force to contract inward in the radial direction by the rigid component 230. The inner surface 223 of the elastic body 220, which is a wall surface of the hollow portion 221, moves radially inward by the external force applied from the rigid component 230. Since the deformation of the outer peripheral surface of the elastic body 220 is restricted by the inner peripheral surface of the rigid component 230, the elastic body 220 is deformed toward the hollow portion 221 of the elastic body 220 having no restriction by the rigid component 230.

As a result, the first member 30 and the second member 40 are pressed inward in the radial direction r by the elastic body 220. While the first member 30 and the second member 40 press each other in the axial direction X, the inner surface 223 which is a wall surface of the hollow portion 221 comes into contact with the first member 30 and the second member 40. Here, the first member 30 and the second member 40 are in the close state, and the rigid component 230 is in the external force application state.

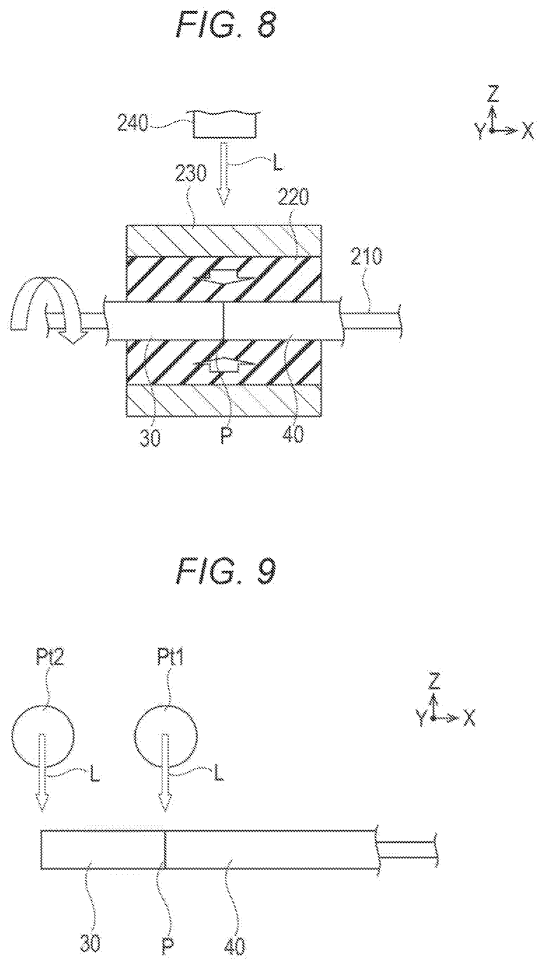

Next, the laser irradiation unit 240 sets the irradiation position of the laser beam on the adjacent portion P between the first member 30 and the second member 40 as illustrated in FIG. 8 by the galvano scan or the like, and the adjacent portion P is irradiated with a laser beam L (S4). In the present embodiment, when the insertion member 210, the elastic body 220, and the rigid component 230 rotate about the axial direction X as a rotation axis, the first member 30 and the second member 40 rotate about the axial direction X as a rotation axis. Thus, a fusion portion is formed from the outer periphery to the inside of the adjacent portion P between the first member 30 and the second member 40 by heat generation of the first member 30 and the second member 40 and/or heat transfer from the insertion member 210 to the first member 30 and the second member 40.

In the present embodiment, not only the adjacent portion P between the first member 30 and the second member 40 as indicated as a position Pt1 in FIG. 9 but also the distal portion of the first member 30 indicated as a position Pt2 in FIG. 9 and different from the adjacent portion P are irradiated with the laser beam L. Thus, the distal portion of the first member 30 is deformed, and the corner portion at the distal portion of the first member 30 is formed smoothly like a curved surface. The position Pt1 and the position Pt2 may be irradiated with the laser beam L intermittently or continuously.

Next, the rigid component 230 is separated from the elastic body 220 (S5). In the present embodiment, the rigid component 230 is moved relative to the elastic body 220 in the axial direction X to release the application of the external force to the elastic body 220 by the rigid component 230. Thus, the wall surface of the hollow portion 221 of the elastic body 220 that has been in contact with the first member 30 and the second member 40 as illustrated in FIG. 7 is separated once again with a gap as illustrated in FIG. 6.

The elastic body 220 may be extended in the axial direction X not only in step S3 but also for separating the elastic body 220 from the rigid component 230 (S5).

Next, the insertion member 210 to which the first member 30 and the second member 40 are attached is moved with respect to the elastic body 220 to remove the first member 30 and the second member 40 joined to each other from the insertion member 210 (S6). Thereafter, an operation of attaching the kink protector 21 and the hub 20 to the catheter body 10 is performed. Thus, the manufacture of the catheter 100 illustrated in FIG. 1 is completed.

As described above, in the manufacturing method for the catheter 100 including the first member 30 and the second member 40 each having a tubular shape according to the present embodiment, the adjacent portion P between the first member 30 and the second member 40 is irradiated with the laser beam L to be fused in the close state and the external force application state.

In the close state, the elastic body 220 having laser transmission properties comes into contact with the first member 30 and the second member 40 in the hollow portion 221 by elastic deformation to bring the first member 30 and the second member 40 close to each other. The elastic body 220 has the hollow portion 221 through which the first member 30 and the second member 40 can be inserted with a gap at no load, and is configured to be elastically deformable.

In the external force application state, the rigid component 230 having laser transmission properties and greater rigidity than the elastic body 220 applies the external force to the elastic body 220 so as to restrain a decrease in force due to the elastic deformation that brings the first member 30 and the second member 40 close to each other.

In addition, the manufacturing apparatus 200 for the catheter 100 includes the insertion member 210 that can be inserted into the first member 30 and the second member 40, and the elastic body 220 and the rigid component 230 which are described above.

With this configuration, when the application of the external force to the elastic body 220 by the rigid component 230 is stopped after the irradiation of the laser beam L, the elastic body 220 that has been in contact with the first member 30 and the second member 40 is separated from the elastic body 220 with a gap. Although a method for fusing using a heat shrinkable tube needs a work for removing the shrunk tube from the first member and the second member after fusion, the method according to the present embodiment can accordingly eliminate the removal work described above.

This can eliminate or reduce the need to redo the removal work due to a failure in removing the tube in the case of a method using a heat shrinkable tube. Since the elastic body 220 returns to a state (state before being used) with a gap relative to the first member 30 and the second member 40 by elastic deformation without being cut, the elastic body 220 can be used for multiple fusions of the first member 30 and the second member 40. This can contribute to labor saving and reduction in material cost.

Since the melted portions of the first member 30 and the second member 40 are formed in the vicinity of the section irradiated with the laser beam L, the form of the melted portion can be widely selected by controlling the section irradiated with the laser beam. Furthermore, when the elastic body 220 having a circular cross section intersecting the axial direction X uniformly pressurizes the outer periphery of the adjacent portion P between the first member 30 and the second member 40 by the rigid component 230 also having a circular cross section intersecting the axial direction X, the quality of the fusion portion can be improved.

At least one of the first member 30 and the second member 40 is configured to include a material that absorbs the laser beam L in the adjacent portion P. With this configuration, the first member 30 and the second member 40 can be effectively fused by heat generation due to the laser beam being absorbed at the adjacent portion P between the first member 30 and the second member 40.

The first member 30 and the second member 40 have a tubular shape, and according to the manufacturing method according to the present embodiment, it is possible to manufacture the catheter 100 in which the second member 40 is disposed on the proximal side in the axial direction X with respect to the first member 30.

In the manufacturing method according to the present embodiment, a portion different from the adjacent portion P in the axial direction X of the first member 30 such as a distal portion is irradiated with the laser beam L, whereby this portion can be formed to have a curved surface or the like.

In the external force application state, the rigid component 230 is disposed outside the elastic body 220 in the radial direction intersecting the axial direction X of the first member 30 and the second member 40 and applies an external force to the elastic body 220. That is, in the external force application state, the rigid component 230 is radially outside the elastic body 220 and axially overlaps the first member 30 and the second member 40 so that the axial extent of the rigid component 230 coincides with an axially extending portion of the first member 30 and an axially extending portion of the second member 40 as shown in FIG. 7. With this configuration, it is possible to restrain separation of the first member 30 and the second member 40 and to fuse the adjacent portion P between the first member 30 and the second member 40.

The rigid component 230 includes the insertion portion N through which the elastic body 220 can be inserted. The outer surface of the elastic body 220 is formed to have an interference fit with respect to the insertion portion N at no load. Due to the rigid component 230 being placed to be fitted to the outer diameter portion of the elastic body 220 in this manner, an external force that brings the first member 30 and the second member 40 close to each other can be applied to the elastic body 220.

The elastic body 220 is disposed inside the insertion portion N of the rigid component 230 with the outer diameter of the elastic body 220 being changed to be smaller than the inner diameter of the rigid component 230 by being extended in the axial direction X. With this configuration, the elastic body 220 can be easily disposed inside the insertion portion N of the rigid component 230.

In the present embodiment, the elastic body 220 maintains a predetermined shape at no load, and is deformed toward the hollow portion 221 of the elastic body 220 having no restriction by the rigid component 230 when an external force is applied by the rigid component 230, so that it is possible to stably pressurize the workpiece such as the first member 30 and the second member 40. In the method according to the present embodiment, the hollow portion 221 of the elastic body 220 can have any shape, so that the workpiece can be processed into any shape.

### Modification of First Embodiment

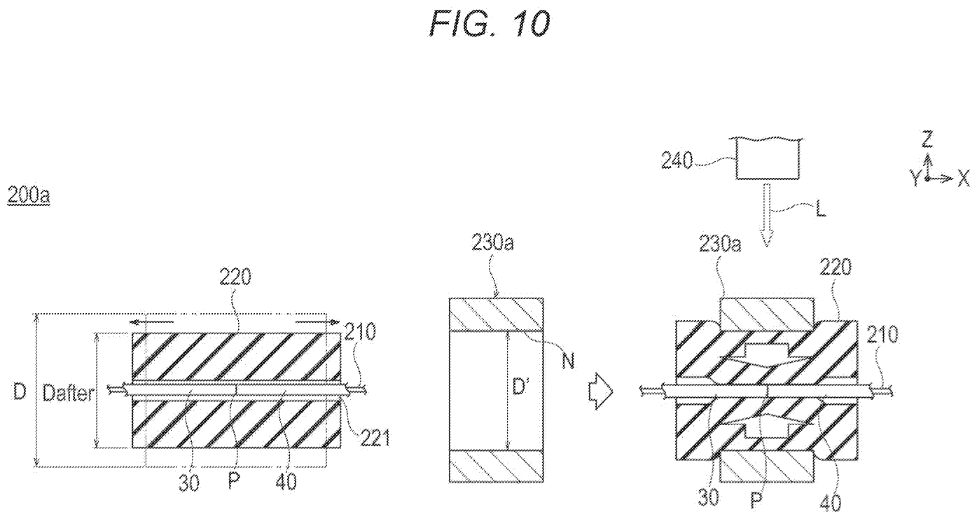

FIG. 10 is a diagram illustrating a manufacturing apparatus 200a for catheters according to a modification of the first embodiment. The first embodiment has described an example in which the length of the rigid component 230 in the axial direction X is the same as the length of the elastic body 220 in the axial direction X. However, the length of the rigid component in the axial direction X is not limited to the above length as long as the rigid component can apply an external force such that the elastic body brings the first member and the second member close to each other in the axial direction X.

In addition to the above configuration, the length of a rigid component 230a in the axial direction X may be shorter than the length of the elastic body 220 in the axial direction X as illustrated in FIG. 10. The configuration of the manufacturing apparatus for catheters other than the rigid component and the manufacturing method for catheters may be similar to those of the first embodiment, and thus, the common description will be omitted.

In the present embodiment, in a state where the elastic body 220 is extended, the elastic body 220 is configured to be longer in the axial direction X than the rigid component 230a at no load. Therefore, in view of the arrangement of the elastic body 220 in the rigid component 230a, a grip is provided on the elastic body 220, so that the elastic body 220 can be easily inserted into and removed from the rigid component.

### Second Embodiment

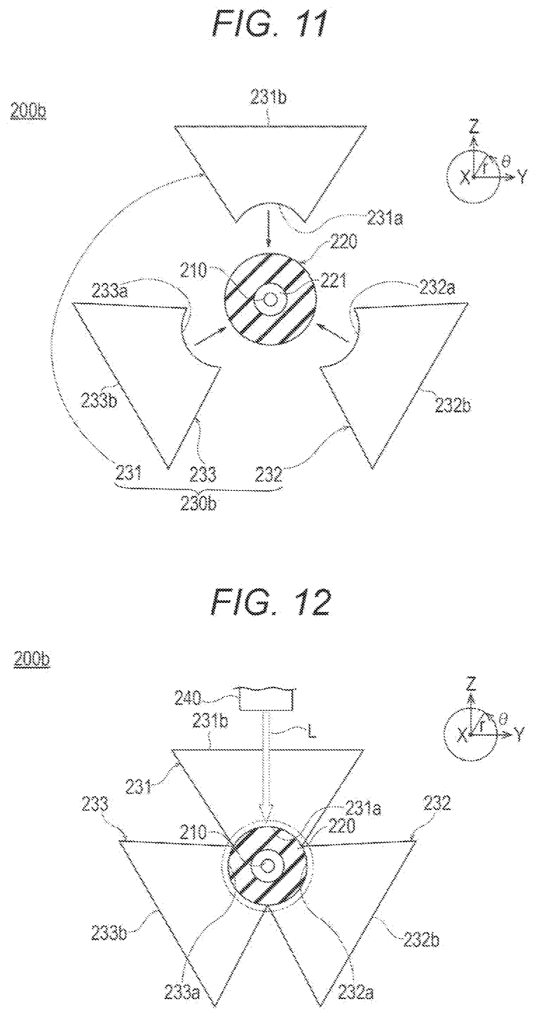

FIG. 11 is a diagram illustrating a state before a rigid component 230b (corresponding to a first rigid component) constituting a manufacturing apparatus 200b for catheters according to the second embodiment applies an external force to the elastic body 220, and FIG. 12 is a diagram illustrating a state in which the rigid component 230b applies an external force to the elastic body 220.

The first embodiment has described an example in which, similar to the elastic body 220, the rigid component 230 is formed in a tubular shape, and the rigid component 230 is attached to the elastic body 220 so as to be fitted to the elastic body 220. However, the rigid component may have the following configuration. In the present embodiment, the insertion member 210, the elastic body 220, and the laser irradiation unit 240 are the same as those in the first embodiment, and thus, the description thereof will be omitted.

FIGS. 11 and 12 include cylindrical coordinates. A letter of “r” of the cylindrical coordinate system indicates a direction in the radial direction or a radially extending direction from the centers of the first member 30 and the second member 40 constituting the catheter 100 along the planar direction YZ, and is referred to as a radial direction r. A letter of “θ” indicates a direction along a circumferential direction or an angular direction of the first member 30, the second member 40, and the like in the planar direction YZ intersecting the axial direction X of the first member 30 and the second member 40, and is referred to as circumferential direction θ.

In the external force application state, the rigid component 230b is disposed outside the elastic body 220 in the radial direction r intersecting the axial direction X of the first member 30 and the second member 40 and applies an external force to the elastic body 220. As illustrated in FIG. 11, the rigid component 230b includes a plurality of components obtained by dividing the rigid component 230b into a predetermined number of sections in the circumferential direction θ intersecting the axial direction X of the first member 30 and the second member 40. The rigid component 230b includes a component 231, a component 232, and a component 233 in the present embodiment.

The component 231, the component 232, and the component 233 are configured to elastically deform the elastic body 220 by applying an external force to the elastic body 220 toward the central axes of the first member 30 and the second member 40 in the external force application state. The component 231, the component 232, and the component 233 are configured to be movable so as to move close to or away from the elastic body 220 in the radial direction r. The component 231, the component 232, and the component 233 have an inner surface 231a, an inner surface 232a, and an inner surface 233a, respectively, as inner surfaces facing the elastic body 220. The component 231, the component 232, and the component 233 have an outer surface 231b, an outer surface 232b, and an outer surface 233b, respectively, as outer surfaces with respect to the central axes.

With this configuration, the elastic body 220 can be separated from the first member 30 and the second member 40 with a gap, or the elastic body 220 can be brought into contact with the first member 30 and the second member 40.

The component 231, the component 232, and the component 233 come into contact with the elastic body 220 so as to surround the outer periphery of the elastic body when coming into contact with the elastic body 220. The radiuses of curvature of the inner surfaces 231a, 232a, and 233a are smaller than the radius of curvature of the outer surface of the elastic body 220 at no load. The inner surfaces 231a, 232a, and 233a form a circle in the planar direction YZ in the external force application state. The formed circular cross-sectional area is smaller than the cross-sectional area in the planar direction YZ of the elastic body 220 at no load. The area of each of the inner surface 231a of the component 231, the inner surface 232a of the component 232, and the inner surface 233a of the component 233 is smaller than the area of the corresponding one of the outer surface 231b, outer surface 232b, and outer surface 233b. The component 231, the component 232, and the component 233 are configured to surround the outer periphery of the elastic body 220 in the circumferential direction θ divided into three at equal angles in the present embodiment.

However, the number of components is not limited to three, and the angles of the components (circumferential angular extent) may not be uniform as long as a pressing force can be generated at the adjacent portion P so that the first member 30 and the second member 40 are not separated from each other during fusion.

Each of the component 231, the component 232, and the component 233 of the rigid component 230b can be synchronously moved in the radial direction r by a hydraulic cylinder (not shown) or the like. Further, the component 231, the component 232, and the component 233 of the rigid component 230b are configured to be rotatable with the axial direction X as a rotation axis when at least the position in the radial direction r is not moved, as in the insertion member 210 and the like of the first embodiment.

Next, a manufacturing method for catheters according to the present embodiment will be described. Note that steps S1, S2, and S6 are the same as those in the first embodiment, and thus the description thereof will be omitted.

After the adjacent portion P between the first member 30 and the second member 40 is placed in the hollow portion 221 of the elastic body 220, the component 231, the component 232, and the component 233 of the rigid component 230b are moved in the radial direction r toward the elastic body 220 and brought into contact with the elastic body 220 as illustrated in FIG. 12. The component 231, the component 232, and the component 233 of the rigid component 230b press the elastic body 220 in the radial direction r. As a result, the elastic body 220 receives an inward external force in the radial direction r by the rigid component 230b (S3), and the elastic deformation of the elastic body 220 causes the first member 30 and the second member 40 to be pressed against each other at the adjacent portion P. The outer diameter of the elastic body 220 at no load indicated by an alternate long and two short dashes line in FIG. 12 is changed to a decreased outer diameter. Since the deformation of the elastic body 220 to the outer surface is restricted by the rigid component 230b, the elastic body 220 is deformed to the hollow portion 221 having no restriction. As a result, the first member 30 and the second member 40 are pressed inward in the radial direction r by the elastic body 220. FIG. 12 does not illustrate the deformation of the hollow portion of the elastic body 220.

In this state, the laser irradiation unit 240 adjusts the irradiation position of the laser beam L to the adjacent portion P, and emits the laser beam L. When the laser beam L is emitted while the first member 30 and the second member 40 rotate due to the rotation of the insertion member 210, the elastic body 220, and the rigid component 230b which is determined in position in the radial direction r about the axial direction X as a rotation axis, a fusion portion is formed on the outer periphery of the adjacent portion P (S4). The laser beam L is emitted to the outer periphery of the adjacent portion P between the first member 30 and the second member 40 through the outer surface 231b, the outer surface 232b, the outer surface 233b, and the inner surfaces 231a, 232a, and 233a of the component 231, the component 232, and the component 233 of the rigid component 230b and the elastic body 220.

After the fusion is completed, the component 231, the component 232, and the component 233 of the rigid component 230b are moved outward in the radial direction r to be separated from the elastic body 220 (S5). Thus, the elastic body 220 that has been in contact with the first member 30 and the second member 40 is separated from the first member 30 and the second member 40 with a gap, as in the first embodiment.

As described above, in the second embodiment, the rigid component 230b includes the plurality of components 231, 232, and 233 distributed in the circumferential direction θ in the external force application state. The plurality of components 231, 232, and 233 is configured to elastically deform the elastic body 220 by applying an external force to the elastic body 220 toward the central axes of the first member 30 and the second member 40.

As described above, the rigid component 230b elastically deforms the elastic body 220 to apply a pressing force so as to prevent the first member 30 and the second member 40 from being separated from each other, and with this state, fusion can be performed at the adjacent portion P, as in the first embodiment.

In the present embodiment, the elastic body 220 maintains a predetermined shape at no load, and is deformed toward the hollow portion 221 of the elastic body 220 having no restriction on deformation by the rigid component 230b when an external force is applied by the rigid component 230b, so that it is possible to stably pressurize the workpiece such as the first member 30 and the second member 40. The method according to the present embodiment enables fine control of pressurization. In the method according to the present embodiment, the hollow portion 221 of the elastic body 220 can have any shape, so that the workpiece can be processed into any shape.

### Modification of Second Embodiment

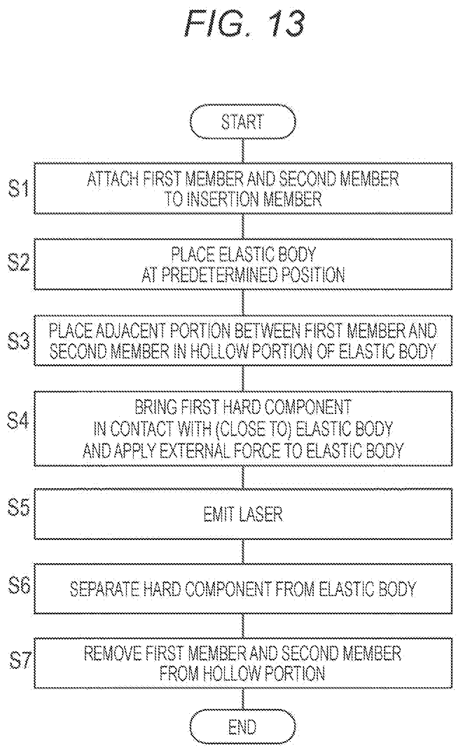

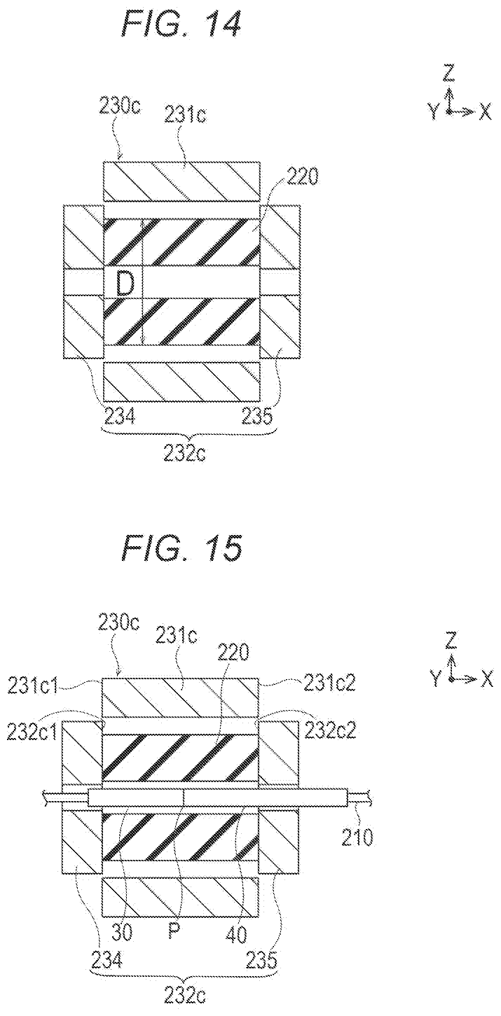

FIG. 13 is a flowchart illustrating a manufacturing method for catheters according to a modification of the second embodiment, and FIGS. 14 to 18 are diagrams for describing a manufacturing apparatus 200c for catheters. The second embodiment has described an example in which the component 231, the component 232, and the component 233 of the rigid component 230b apply an external force to the elastic body 220 from the outside to the inside in the radial direction r of the first member 30 and the second member 40.

However, the rigid component may have the following configuration. The insertion member 210, the elastic body 220, and the laser irradiation unit 240 are the same as those in the first embodiment, and thus, the description thereof is not repeated.

As illustrated in FIG. 14 and the like, a rigid component 230c includes a first rigid component 231c and a second rigid component 232c. In the external force application state, the first rigid component 231c is disposed outside the elastic body 220 in the radial direction r intersecting the axial direction X of the first member 30 and the second member 40. The first rigid component 231c applies an external force to the elastic body 220. Since the first rigid component 231c includes the component 231, the component 232, and the component 233 described in the second embodiment, the description of the component 231, the component 232, and the component 233 will not be repeated.

The second rigid component 232c applies an external force to the elastic body 220 from a direction different from that of the first rigid component 231c. The second rigid component 232c is disposed outside the elastic body 220 in the axial direction X different from the radial direction r in which the first rigid component 231c applies an external force in the present modification. The second rigid component 232c applies an external force to the elastic body 220.

The second rigid component 232c includes a component 234 and a component 235, and the component 234 and the component 235 are formed into a substantially cylindrical shape so as to surround the elastic body 220 from the outside in the axial direction X. The component 234 and the component 235 of the second rigid component 232c are disposed with the surface on the elastic body 220 side in contact with the side surface of the elastic body 220. With this configuration, the second rigid component 232c can cover the outer surface of the elastic body 220 together with the first rigid component 231c. In addition, similar to the insertion member 210 or the like in the first embodiment, the second rigid component 232c is rotatably supported with the axial direction X as a rotation axis. Further, the component 234 and the component 235 are configured to be movable in the axial direction X so that the elastic body 220 can be disposed or positioned.

Next, the manufacturing method for the catheter 100 according to the present modification will be described with reference to FIG. 13. In the present modification, step S1 in the flowchart of FIG. 13 is similar to step S1 in the flowchart of FIG. 4, and thus the description thereof will not be repeated.

After the first member 30 and the second member 40 are attached to the insertion member 210, the elastic body 220 is placed in the apparatus at a position where the fusion portion is to be formed (S2). Here, the elastic body 220 is disposed at a position where the fusion portion is to be formed in a state where the second rigid component 232c is in contact with the elastic body 220 in advance in the axial direction X as illustrated in FIG. 14. Next, the adjacent portion P between the first member 30 and the second member 40 is placed in the hollow portion 221 of the elastic body 220 (S3, see FIG. 15) as in step S2 in FIG. 4.

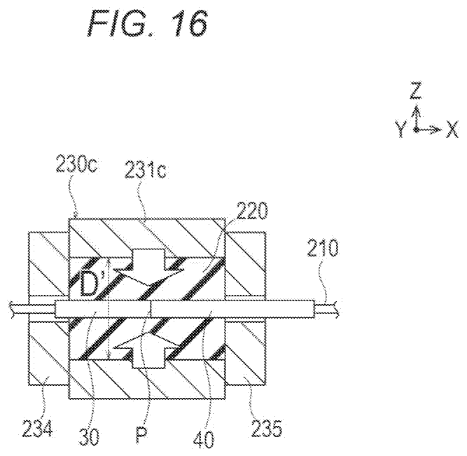

Next, the component 231, the component 232, and the component 233 of the first rigid component 231c are moved in the radial direction r toward the elastic body 220 and brought into contact with the elastic body 220 (outer surface of the elastic body 220) as illustrated in FIG. 16. That is, the outer side surface of one of the first rigid component 231c and the second rigid component 232c moves along the inner side surface of the other. Referring to FIG. 15, an outer side surface 231c1 which is one of the outer side surfaces and an outer side surface 231c2 which is the other outer side surface of the first rigid component 231c are moved along an inner side surface 232c1 which is one of the inner side surfaces and an inner side surface 232c2 which is the other inner side surface of the second rigid component 232c, respectively. Then, an external force is applied to the elastic body 220 by the first rigid component 231c and the second rigid component 232c. Thus, the external force is applied to the elastic body 220 in the radial direction r and the axial direction X by the first rigid component 231c and the second rigid component 232c.

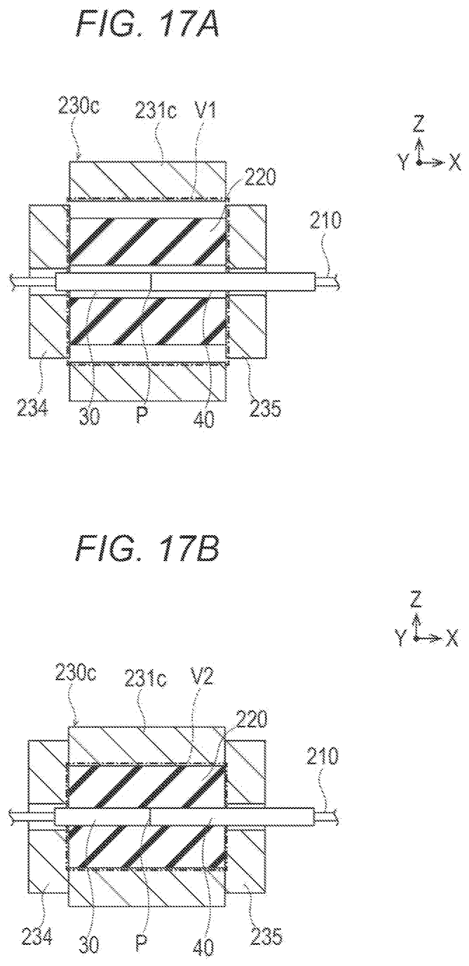

As illustrated in FIGS. 17A and 17B, the internal volume defined by the first rigid component 231c and the second rigid component 232c decreases. Specifically, as indicated by an alternate long and short dash line in FIG. 17A, the internal volume defined by the first rigid component 231c and the second rigid component 232c at no load is defined as an internal volume V1. On the other hand, as indicated by an alternate long and short dash line in FIG. 17B, the internal volume defined by the first rigid component 231c and the second rigid component 232c in the external force application state is defined as an internal volume V2. The internal volume defined by the first rigid component 231c and the second rigid component 232c decreases from the internal volume V1 to the internal volume V2. As a result, the elastic body 220 transitions from the no load state to the external force application state. The surface of the elastic body 220 is surrounded, so that the volume of the elastic body 220 decreases (see D in FIG. 14 and D′ in FIG. 16, D>D′). As described above, the internal volume defined by the first rigid component 231c and the second rigid component 232c decreases, whereby the volume of the elastic body 220 decreases. Accordingly, the elastic body 220 transitions from the no load state to the external force application state (S4). In addition, the first member 30 and the second member 40 are in the close state due to application of an external force to the elastic body 220 by the rigid component 230c.

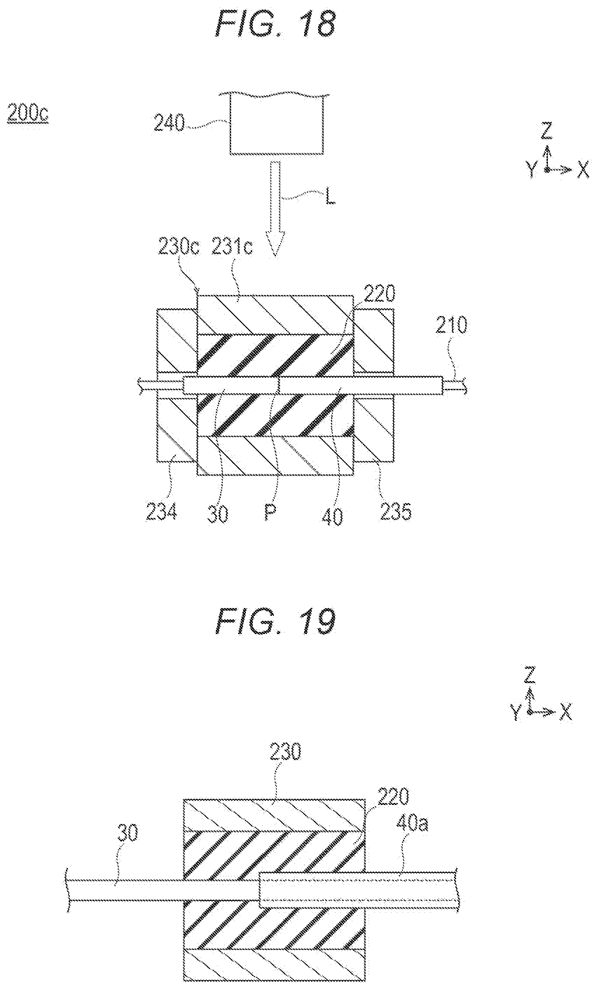

In this state, the laser beam L is transmitted through the rigid component 230c and the elastic body 220 and is applied to the adjacent portion P between the first member 30 and the second member 40, as illustrated in FIG. 18. When the first member 30 and the second member 40 rotate due to the rotation of the insertion member 210, the elastic body 220, and the rigid component 230c about the axial direction X as a rotation axis, a fusion portion is formed on the outer periphery of the adjacent portion P (S5).

After the fusion, the first rigid component 231c and the second rigid component 232c are separated from the elastic body 220. That is, the component 231, the component 232, and the component 233 constituting the first rigid component 231c are moved outward in the radial direction r, and the component 234 and the component 235 constituting the second rigid component 232c are moved outward in the axial direction X. The order of separation of the first rigid component 231c and the second rigid component 232c is not particularly limited. As a result, the rigid component 230c is separated from the elastic body 220 (S6).

Then, the first member 30 and the second member 40 which are fused to each other are removed from the hollow portion 221 of the elastic body 220 (S7), as in step S6 in FIG. 4. Then, the hub 20 and the kink protector 21 are attached to form a catheter.

As described above, in the present modification, the rigid component 230c includes the first rigid component 231c and the second rigid component 232c. The first rigid component 231c is disposed outside the elastic body 220 in the radial direction r, and applies an external force to the elastic body 220 in the radial direction r. The second rigid component 232c is disposed outside the elastic body 220 in the axial direction X of the first member 30 and the second member 40, and applies an external force to the elastic body 220 in the axial direction X.

By using the first rigid component 231c and the second rigid component 232c as described above, it is possible to limit unnecessary deformation of the elastic body 220 that is not related to fusion of the first member 30 and the second member 40.

Thus, an amount of compression of the elastic body 220 can be reduced, and the compression of the elastic body can be easily controlled. In addition, the positional displacement of the first member 30 and the second member 40 can be prevented or limited, whereby dimensional accuracy of the finished catheter 100 can be improved.

As the internal volume defined by the first rigid component 231c and the second rigid component 232c decreases, the elastic body 220 transitions from the no load state to the external force application state. As a result, it is possible to prevent or limit an unintended deformation of the elastic body 220 that could occur if an external force is applied from one direction, and to prevent or restrain a decrease in dimensional accuracy of the finished catheter.

The present invention is not limited only to the embodiments and the modifications thereof described above, and various changes can be made within the scope of the claims. FIG. 19 is a diagram illustrating a modification of the first member and the second member. The method for fusing the first member 30 and the second member 40 adjacent to each other in the axial direction X has been described above.

However, the direction in which the first member and the second member are arranged is not limited to the axial direction X, and they may be arranged in the other directions. For example, the first member 30 and a second member 40a which are arranged in the radial direction as illustrated in FIG. 19 may be fused by a manufacturing apparatus including an insertion member, an elastic body, a rigid component, and a laser irradiation unit.

Even when the members to be fused such as the first member 30 and the second member 40a are arranged in the radial direction, the laser irradiation unit irradiates a close portion, which is the fusion portion, with a laser beam from outside in the radial direction intersecting with the axial direction of the first member 30 and the second member 40a, like the laser irradiation unit 240.

In the present embodiment, the elastic body 220 maintains a predetermined shape at no load, and is deformed toward the hollow portion 221 of the elastic body 220 having no restriction by the first rigid component 231c and the second rigid component 232c when an external force is applied, so that it is possible to stably pressurize the workpiece such as the first member 30 and the second member 40. The method according to the present embodiment can finely control pressurization, and further has quick responsiveness of pressurization. Further, in the method according to the present embodiment, the hollow portion 221 of the elastic body 220 can have any shape, so that the workpiece can be processed into any shape.

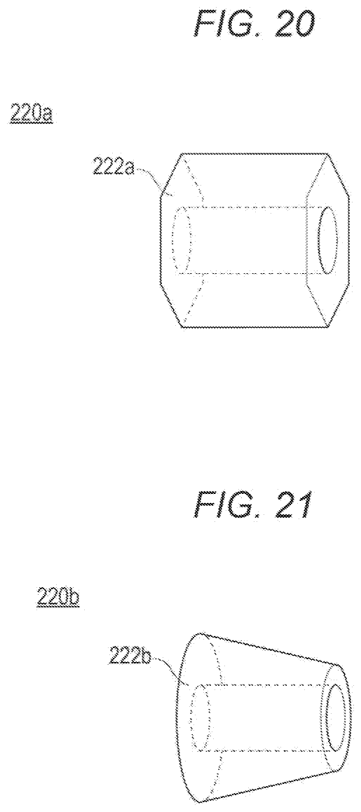

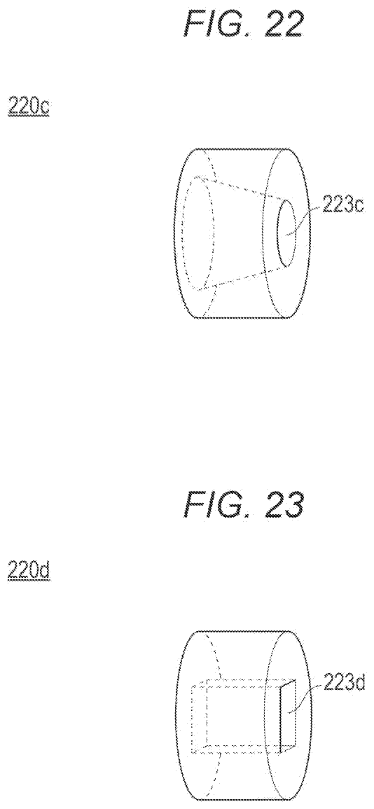

FIGS. 20 and 21 are diagrams illustrating a modification of the outer surface of the elastic body, and FIGS. 22 and 23 are diagrams illustrating a modification of the inner surface of the elastic body. Although the first embodiment is an example in which the outer surface 222 and the inner surface 223 of the elastic body 220 are the side surfaces of the cylinder in FIG. 3, the elastic body 220 is not limited to that configuration. Instead of the above configuration, an outer surface 220a of an elastic body 222a may be formed as a side surface of a hexagonal prism as an example of a polygonal prism as illustrated in FIG. 20, or an outer surface 220b of an elastic body 222b may be formed as a side surface of a truncated cone as illustrated in FIG. 21.

In addition, an inner surface 220c of an elastic body 223c may be formed as a side surface of a truncated cone as illustrated in FIG. 22, or an inner surface 220d of an elastic body 223d may be formed as a side surface of a quadrangular prism which is an example of a polygonal prism as illustrated in FIG. 23. When the cross sections intersecting (orthogonal to) the axial direction X of the rigid component and the elastic body are only circular, the first member and the second member can be uniformly pressurized at any position in the circumferential direction over the entire circumference.

In addition, the first embodiment is an example in which the laser beam L is applied to the position Pt2 corresponding to the distal portion of the first member 30 in addition to the position Pt1 corresponding to the adjacent portion P between the first member 30 and the second member 40 as illustrated in FIG. 9. However, a case where the position Pt1 is irradiated with the laser beam L and the position Pt2 is not irradiated with the laser beam L is also included in the scope of the disclosure here.

In the above description, the first member and the second member are rotated with the axial direction X as a rotation axis by rotating the insertion member, the elastic body, and the rigid component to form the fusion portion on the outer periphery of the adjacent portion P. However, the configuration is not limited thereto, and the laser irradiation unit may change the irradiation position or the irradiation direction of a laser beam without rotating the insertion member, the elastic body, and the rigid component to form the fusion portion on the outer periphery of the adjacent portion P between the first member and the second member.

The modification of the second embodiment is an example in which the second rigid component 232c is in contact with the elastic body 220 in advance when the elastic body 220 is placed at the position where the fusion portion is to be formed in step S2 of FIG. 13. However, the configuration is not limited thereto, and the second rigid component 232c may be brought close to the elastic body 220 after the elastic body 220 is placed at the position where the fusion portion is to be formed.

The detailed description above describes embodiments and modifications of a catheter manufacturing method and catheter manufacturing apparatus representing examples of the new catheter manufacturing method and catheter manufacturing apparatus disclosed here. The invention is not limited, however, to the precise embodiments, modifications and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A manufacturing method for manufacturing a catheter including a catheter body formed by fusing a first member and a second member, the manufacturing method comprising
irradiating an adjacent portion between the first member and the second member with a laser beam to fuse the adjacent portion in a close state and in an external force application state, the close state being a state in which an elastically deformable elastic body having laser transmission property and having a hollow portion in which the first member and the second member are positioned is in contact with the first member and the second member in the hollow portion due to elastic deformation of the elastic body, the external force application state being a state in which a component having the laser transmission property and having a rigidity greater than the elastic body applies an external force to the elastic body to limit a decrease in force due to the elastic deformation for bringing the first member and the second member close to each other.

2. The manufacturing method for manufacturing a catheter according to claim 1, wherein at least one of the first member and the second member includes a material that absorbs a laser beam in the adjacent portion.

3. The manufacturing method for manufacturing a catheter according to claim 1, wherein
the first member and the second member have a tubular shape, and
the second member is disposed on a proximal side in an axial direction with respect to the first member.

4. The manufacturing method for manufacturing a catheter according to claim 3, wherein
the first member includes one end and an opposite end, the one end of the first member being positioned adjacent the second member in the axial direction, and
the laser beam is also applied to the opposite end of the first member.

5. The manufacturing method for a catheter according to claim 1, wherein
the first member and the second member have a tubular shape, and
the component includes a first component that is disposed outside the elastic body in a radial direction intersecting an axial direction of the first member and the second member and applies the external force to the elastic body in the external force application state.

6. The manufacturing method for a catheter according to claim 1, wherein
the component includes an insertion portion in which the elastic body is positioned, the elastic body being positioned in the insertion portion before the irradiation by virtue of the elastic body being inserted in an inserting direction into the insertion portion, and
the elastic body has an outer surface extending in the insertion direction of the elastic body, the outer surface of the elastic body having an interference fit with the insertion portion of the component in the no load state.

7. The manufacturing method for a catheter according to claim 6, wherein
the first member and the second member have a tubular shape, and
before the irradiating of the adjacent portion, the elastic body is placed inside the insertion portion of the component with an outer diameter of the elastic body being reduced to be smaller than an inner diameter of the component by virtue of the elastic body being extended in an axial direction of the first member and the second member.

8. The manufacturing method for a catheter according to claim 1, wherein
the component includes a plurality of components positioned adjacent one another in a circumferential direction in the external force application state, and
the plurality of components applies the external force to the elastic body toward central axes of the first member and the second member to elastically deform the elastic body in the external force application state.

9. The manufacturing method for a catheter according to claim 5, wherein the component includes a second component that is disposed outside the elastic body in the axial direction of the first member and the second member and applies the external force to the elastic body.

10. The manufacturing method for a catheter according to claim 9, wherein the no load state is shifted to the external force application state by decreasing an internal volume defined by the first component and the second component.

11. A manufacturing apparatus for a catheter, the manufacturing apparatus comprising:
an insertion member insertable into the first member and the second member; and
the elastic body and the component according to claim 1.

12. A manufacturing method for manufacturing an elongated medical device, the manufacturing method comprising:
disposing a tubular member in a hollow portion of an elastic body, the tubular body extending in an axial direction from one axial end of the tubular member to an opposite axial end of the tubular member, the disposing of the tubular member in the hollow portion of the elastic body including disposing the tubular member in the hollow portion of the elastic body so that an end portion of the tubular member, inclusive of the one axial end of the tubular member, is located in the hollow portion of the elastic body, the elastic body having laser transmission property;
applying an external force to the elastic body by at least one of two components, the two components being a first component and a second component that are both positioned outside the elastic body, the first component being positioned radially outside the elastic body in a radial direction that intersects the axial direction, the second component being positioned axially outside the elastic body in the axial direction, the first component having laser transmission property, the at least one of the two components that applies the external force to the elastic body being in contact with the elastic body during the applying of the external force; and
emitting a laser beam through the first component and the elastic body to the distal portion of the tubular member that is located in the hollow portion of the elastic body to deform the distal portion of the tubular member, the laser beam having a wavelength that generates heat by radiation heating in the distal portion of the tubular member.

13. The manufacturing method for a catheter according to claim 12, wherein the disposing of the tubular member in the hollow portion of the elastic body includes disposing the tubular member in the hollow portion of the elastic such that a radially inwardly facing inner surface of the elastic body is spaced from a radially outwardly facing outer surface of the distal portion of the tubular member.

14. The manufacturing method for a catheter according to claim 13, wherein the applying of the external force to the elastic body by the at least one of the two components causes the radially inwardly facing inner surface of the elastic body to move into contact with the radially outwardly facing outer surface of the distal portion of the tubular member.

15. The manufacturing method for a catheter according to claim 12, wherein before the applying of the external force to the elastic body by the at least one of the two components, a part of the at least one of the two components is spaced from the elastic component, the applying of the external force to the elastic body by the at least one of the two components including moving the part of the at least one of the two components towards the elastic body to move the part of the at least one of the two components into contact with the elastic body.

16. A manufacturing method for manufacturing an elongated medical device, the manufacturing method comprising:
positioning an elastic body and a tubular member relative to one another so that a distal portion of the tubular member is located in a hollow portion of the elastic body and an outer surface of the tubular member faces an inner surface of the elastic body, the elastic body having laser transmission property, the tubular member extending in an axial direction;
positioning a component relative to the elastic body so that an inner surface of the component faces an outer surface of the distal portion of the tubular member, the component having laser transmission property;
applying an external force to the elastic body to press the inner surface of the elastic body to the outer surface of the distal portion of the tubular member; and
irradiating the distal portion of the tubular member with a laser beam by virtue of the laser beam passing through the component and the elastic body, the irradiating of the distal portion of the tubular member being performed to generate heat, by radiation heating, in the distal portion of the tubular member.

17. The manufacturing method for a catheter according to claim 16, further comprising, before the positioning of the elastic body and the tubular member relative to one another so that a distal portion of the tubular member is located in a hollow portion of the elastic body, inserting an insertion member into the tubular member so that the insertion member extends axially beyond opposite ends of the tubular member, the positioning of the elastic body and the tubular member including positioning the elastic body and the tubular member with the insertion member relative to one another so that the distal portion of the tubular member is located in the hollow portion of the elastic body.

18. The manufacturing method for a catheter according to claim 16, wherein the tubular member is a first tubular member, the positioning of the elastic body and the tubular member relative to one another including positioning the elastic body, the first tubular member and a second tubular member relative to one another so that the distal portion of the first tubular member and a proximal portion of the second tubular member are located in the hollow portion of the elastic body.

19. The manufacturing method for a catheter according to claim 16, wherein the positioning of the elastic body and the tubular member relative to one another comprises positioning the elastic body and the tubular member relative to one another so that the outer surface of the tubular member is spaced from the inner surface of the elastic body, and the applying of the external force to the elastic body causing the outer surface of the tubular member and the inner surface of the elastic body to contact one another.

20. The manufacturing method for a catheter according to claim 16, further comprising rotating the tubular member, the elastic body and the component during the irradiating of the distal portion of the tubular member with the laser beam.
